(19) United States
(12) Patent Application Publication
(10) Pub. No.: US 2020/0121878 A1
(43) Pub. Date: Apr. 23, 2020

(54) NOSE TO TRAGUS LENGTH BASED (NTLB) TAPE

(71) Applicants: Lionel Newman, JR., Los Angeles, CA (US); Phyllis Ann Brooks-Newman, Los Angeles, CA (US); Albert Crawford, JR., Power Springs, GA (US); Chanan Nicole Kennedy, Marietta, GA (US); William Kennedy, Marietta, GA (US); Jourdon Karim Rahming, Anaheim, CA (US)

(72) Inventors: Lionel Newman, JR., Los Angeles, CA (US); Phyllis Ann Brooks-Newman, Los Angeles, CA (US); Albert Crawford, JR., Power Springs, GA (US); Chanan Nicole Kennedy, Marietta, GA (US); William Kennedy, Marietta, GA (US); Jourdon Karim Rahming, Anaheim, CA (US)

(21) Appl. No.: 16/654,810

(22) Filed: Oct. 16, 2019

Related U.S. Application Data
(60) Provisional application No. 62/747,891, filed on Oct. 19, 2018.

(51) Int. Cl. A61M 16/04; G01B 3/10

(52) U.S. Cl. CPC A61M 16/04; G01B 3/10; A61M 2209/04; A61M 2210/0618; A61M 2210/0662; A61M 2205/6081

(57) ABSTRACT

Techniques regarding a nose to tragus length measuring tape for determining an endotracheal tube insertion depth are provided. For example, in one or more embodiments a measuring tape is provided, which can comprise an indicium representing an insertion depth of an endotracheal tube based upon a direct correlation between a nasal to tragus length of a patient and the insertion depth.

### CROSS-REFERENCE TO RELATED APPLICATIONS

This application claims priority to U.S. Provisional Patent Application Ser. No. 62/747,891 filed on Oct. 19, 2018, entitled “NOSE TO TRAGUS LENGTH BASED (NTLB) TAPE.” The entirety of the aforementioned application is incorporated by reference herein.

### BACKGROUND

The subject disclosure relates to a nose to tragus length measuring tapes and more specifically, to measuring tapes that can correlate a patient's nasal to tragus length (“NTL”) to the insertion depth for an endotracheal tube (“ETT”) for patients ranging in age from newborn (e.g., including premature infant) to pediatric children of different weights and ages. Additionally, the measuring tapes described herein can comprise alternative reference formulas/charts for estimating ETT insertion depth as well equipment and/or supplies information felt to be of value for respiratory emergencies.

Proper ETT placement is critical to reducing the risk of mainstem intubation, airway trauma, pneumothorax, localized pulmonary interstitial emphysema and accidental dislodgment. Estimators for depth of ETT placement in neonates have conventionally relied on the association between patient weight and/or gestational age and the distance to the mid-trachea. Systems such as the “7-8-9 rule,” where the infant's weight is rounded to the nearest kilogram (kg) and then added to 6 centimeters (cm) to estimate ETT insertion depth, perform reasonably well for moderately preterm infants but can be inaccurate for extremely preterm infants and larger term infants. Weight or gestational age-based tables can improve estimates but are cumbersome, require knowledge of the patient that may not be readily available in an emergency and/or can still lead to improver placement in about 8% of infants.

The Neonatal Resuscitation Program (“NRP”) is an educational program jointly sponsored by the American Academy of Pediatrics (“AAP”) and the American Heart Association (“AHA”). Prior editions (e.g., copyright 2011 and earlier) adopted Tochen's weight-based formula (e.g., 6 cm+weight in kg rounded to the closest Integra) because of its ease of memorization and/or reasonable safety, but this formula can lead to incorrect ETT placement in 40% of all neonates and/or 83% of the extremely low birth weight infants.

The 7^{th} edition (e.g., copyright 2016) of NRP addresses this concern by recommending three potential methods for estimating proper ETT placement. However, it leaves to the individual caregivers to decide which of these three methods to utilize. The first method is to use printed depth markers to place the ETT 1 to 2 cm below vocal cords. The second method is an update to the 6^{th} edition (e.g., copyright 2011) gestational age/weight-based chart in which Tochen's formula is now rounded to the nearest 0.5 cm mark. The third method estimates proper ETT depth based on the NTL+1 cm formula, where NTL is measured as the distance between the base of the nasal septum and the tragus of the ear.

Use of the NTL+1 cm formula to estimate the depth of ETT insertion can lead to appropriate placement in 90% of patients over a broad range of gestational ages and weights. Further, the NTL+1 cm estimate can perform well for infants weighing greater than or equal to 2.5 kg and an NTL+0.5 cm estimate performed well for infants weighing less than 2.5, both predicting the correct ETT position as verified by chest X-ray confirmation. The 7^{th} edition of NRP now endorses NTL-based estimates of neonatal ETT depth specially using the NTL+1 cm formula.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, apparatuses and/or methods regarding determining the insertion depth for an ETT are described.

According to an embodiment, a measuring tape is provided. The measuring tape can comprise an indicium representing an insertion depth of an endotracheal tube based upon a direct correlation between a nasal-tragus length of a patient and the insertion depth.

According to an embodiment, a system is provided. The system can comprise a memory that stores computer executable components. The system can also comprise a processor, operably coupled to the memory, and that can execute the computer executable components stored in the memory. The computer executable components can comprise an insertion depth component that determines an endotracheal tube insertion depth by digitally measuring a nose to tragus length of a patient via an analysis of image data that characterizes an anatomy of the patient.

According to an embodiment, a computer-implemented method is provided. The computer-implemented method can comprise determining, by a system operatively coupled to a processor, an endotracheal tube insertion depth by digitally measuring an NTL of a patient via an analysis of image data that characterizes an anatomy of the patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a diagram of an example, non-limiting measurement side of a measuring tape that can facilitate one or more correlations between a premature, neonatal or infant patient's NTL to an insertion depth for an ETT in accordance with one or more embodiments described herein.

FIG. 2 illustrates a diagram of an example, non-limiting measurement side of a measuring tape that can facilitate one or more correlations between a premature, neonatal or infant patient's NTL to an insertion depth for an ETT in accordance with one or more embodiments described herein.

FIG. 3 illustrates a diagram of an example, non-limiting reference side of a measuring tape that can facilitate one or more correlations between a premature, neonatal or infant patient's NTL to an insertion depth for an ETT in accordance with one or more embodiments described herein.

FIG. 4 illustrates a diagram of an example, non-limiting reference side of a measuring tape that can facilitate one or more correlations between a premature, neonatal or infant patient's age and/or weight to an ETT insertion depth based on an estimated gestational age (“EGA”)/weight based table and/or the 7-8-9 weight based rule in accordance with one or more embodiments described herein.

FIG. 5 illustrates a diagram of another example, non-limiting measurement side of an NTL based measuring tape that can comprise a starting point that can be used in right or left face side measurements and can facilitate one or more correlations between a premature, neonatal or infant patient's NTL to an ETT insertion depth in accordance with one or more embodiments described herein. Additionally, the NTL based measuring tape can be folded along the starting point to form a four-sided tape (e.g., forming two measurement surfaces, comprising one or more indicia, facing away from each other in the folded orientation and two inside surfaces, such as reference surfaces comprising reference information, facing toward each other in the folded orientation).

FIG. 6 illustrates a diagram of another example, non-limiting measurement side of an NTL based measuring tape that can comprise a horizontal folding line differentiating various indicia arrangement schemes that can facilitate one or more correlations between a premature, neonatal or infant patient's NTL to an ETT insertion depth in accordance with one or more embodiments described herein. Additionally, the NTL based measuring tape can be folded along the horizontal folding line to form a four-sided tape (e.g., forming two measurement surfaces, comprising one or more indicia, facing away from each other in the folded orientation and two inside surfaces, such as reference surfaces comprising reference information, facing toward each other in the folded orientation).

FIG. 7 illustrates a diagram of another example, non-limiting measurement side of an NTL based measuring tape for infants of various weight categories that can comprise a horizontal folding line differentiating respective indicia arrangement schemes associated with the various weight categories to facilitate correlations between a premature, neonatal or infant patient's NTL to an ETT insertion depth in accordance with one or more embodiments described herein. The indicia arrangement schemes can comprise multiple rows of indicia scaling in opposing directions to facilitate left or right face side measurements of the infants. Additionally, the NTL based measuring tape can be folded along the horizontal folding line to form a four-sided tape (e.g., forming two measurement surfaces, comprising one or more indicia, facing away from each other in the folded orientation and two inside surfaces, such as reference surfaces comprising reference information, facing toward each other in the folded orientation).

FIG. 8 illustrates a diagram of another example, non-limiting measurement side of an NTL based measuring tape that can comprise a horizontal folding line differentiating indicia arrangement schemes for left or right face side measurements to facilitate one or more correlations between a premature, neonatal or infant patient's NTL to an ETT insertion depth in accordance with one or more embodiments described herein. Additionally, the NTL based measuring tape can be folded along the horizontal folding line to form a four-sided tape (e.g., forming two measurement surfaces, comprising one or more indicia, facing away from each other in the folded orientation and two inside surfaces, such as reference surfaces comprising reference information, facing toward each other in the folded orientation).

FIG. 9A illustrates a diagram of another example, non-limiting measurement side of an NTL based measuring tape that can comprise a horizontal folding line differentiating indicia arrangement schemes for left or right face side measurements to facilitate one or more correlations between a premature, neonatal or infant patient's NTL to an ETT insertion depth in accordance with one or more embodiments described herein. Additionally, the NTL based measuring tape can comprise a coding scheme that can identify different types and/or sizes of ETT securing devices using color-code bars associated with the patient's measured NTL.

FIG. 9B illustrates a diagram of another example, non-limiting measurement side of an NTL based measuring tape that can comprise a horizontal folding line differentiating indicia arrangement schemes for left or right face side measurements to facilitate one or more correlations between a premature, neonatal or infant patient's NTL to an ETT insertion depth in accordance with one or more embodiments described herein. Additionally, the NTL based measuring tape can comprise a coding scheme that can identify different types and/or sizes of ETT securing devices using color-code bars associated with the patient's measured NTL.

FIG. 10 illustrates a diagram of another example, non-limiting measurement side of an NTL based measuring tape that can comprise one or more notation areas that can be used to annotate one or more correlations between a premature, neonatal or infant patient's NTL to an ETT insertion depth in accordance with one or more embodiments described herein. Additionally, the NTL based measuring tape can be folded along a horizontal folding line to form a four-sided tape (e.g., forming two measurement surfaces, comprising one or more indicia, facing away from each other in the folded orientation and two inside surfaces, such as reference surfaces comprising reference information, facing toward each other in the folded orientation).

FIG. 11 illustrates a diagram of an example, non-limiting EGA table that can be included on the reference side or measurement side of a measuring tape that can facilitate one or more correlations between a patient's age, weight and/or size to an ETT insertion depth in accordance with one or more embodiments described herein.

FIG. 12 illustrates a diagram of an example, non-limiting table that can be included on the reference side or measurement side of a measuring tape that can facilitate one or more correlations between a patient's weight and the size of an ETT and/or catheter in accordance with one or more embodiments described herein.

FIG. 13 illustrates a diagram of an example, non-limiting reference side of a measuring tape that can facilitate one or more correlations between a patient's age and/or size and an ETT insertion depth along with information regarding the equipment (e.g., suction catheter, etc.) utilized for the insertion in accordance with one or more embodiments described herein.

FIG. 14 illustrates a diagram of an example, non-limiting table of estimation formulas that can be included on the reference side or measurement side of a measuring tape that can facilitate one or more correlations between a patient's age and type of ETT to an insertion depth for the ETT in accordance with one or more embodiments described herein.

FIG. 15 illustrates a diagram of an example, non-limiting table regarding pediatric emergency resuscitation equipment that can be included on the reference side or measurement side of a measuring tape that can facilitate one or more correlations between a patient's age, weight and/or size and the equipment (e.g., pediatric emergency, etc.) utilized for insertion of an ETT in accordance with one or more embodiments described herein.

FIG. 16 illustrates a diagram of another example, non-limiting table that can be included on the reference side or measurement side of a measuring tape that can facilitate one or more correlations between a pediatric patient's age and the equipment (e.g., laryngoscope blade size, etc.) utilized for insertion depth of an ETT in accordance with one or more embodiments described herein

FIG. 17 illustrates a diagram of an example, non-limiting table that can be included on the reference side or measurement side of a measuring tape that can facilitate one or more correlations between a patient's age, weight and/or size and the equipment (e.g., end-tidal carbon dioxide detector, etc.) utilized for insertion of an ETT in accordance with one or more embodiments described herein.

FIG. 18 illustrates a diagram of an example, non-limiting table that can be included on the reference side or measurement side of a measuring tape that can facilitate one or more correlations between a patient's age, weight and/or size and the equipment (e.g., face mask size, etc.) utilized for insertion of an ETT in accordance with one or more embodiments described herein.

FIG. 19 illustrates a diagram of an example, non-limiting table that can be included on the reference side or measurement side of a measuring tape that can facilitate one or more correlations between a patient's age, weight and/or size and the equipment (e.g., tortle infant head positioner, etc.) utilized for insertion of an ETT in accordance with one or more embodiments described herein.

FIG. 20 illustrates a diagram of an example, non-limiting table that can be included on the reference side or measurement side of a measuring tape that can facilitate one or more correlations between a patient's age, weight and/or size and the equipment (e.g., RAM Cannula, etc.) utilized after removal of an inserted ETT in accordance with one or more embodiments described herein.

FIG. 21 illustrates a flow diagram of an example, non-limiting method that can facilitate determining an insertion depth for an ETT using an NTL based measuring tape in accordance with one or more embodiments described herein.

FIG. 22 illustrates a block diagram of an example, non-limiting system that can facilitate one or more correlations between a premature, neonatal or infant patient's NTL to an insertion depth for an ETT in accordance with one or more embodiments described herein.

FIG. 23 illustrates a flow diagram of an example, non-limiting method that can facilitate determining an insertion depth for an ETT using a system that can optically determine a patient's NTL in accordance with one or more embodiments described herein.

FIG. 24 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

When a patient (e.g., an infant, child, or adult) is having difficulty breathing and/or is under-going surgery, a breathing tube can be placed into the patient's mouth or nose to assist their breathing. In the majority of such instances, the breathing tube is placed in the patient's mouth and extends to their lungs. Said breathing tube is referred to as an endotracheal tube (“ETT”), wherein the ETT can be positioned within the patient's trachea. Conventional ETTs have one or more calibrated markings in centimeters (cm) located along a length of the ETTs. Further, ETTs utilized for adults can further comprise a cuff located at a distal end of the ETTs to facilitate creation of a seal once inserted into the patient. ETTs utilized for infant patients can be characterized as having a smaller diameter (e.g., having an outside diameter ranging from greater than or equal to 2.5 millimeters (mm) and less than or equal to 4.0 mm) and/or a shorter length (e.g., ranging from greater than or equal to 13 cm and less than or equal to 19 cm) than ETTs used for adult patients. Also, ETTs used for infant patients can lack a cuff at least because the size of the infant's autonomy renders the existence of a cuff impractical and/or unnecessary.

During intubation of a patient, the ETT can be inserted into the patient's mouth, between the patient's vocal cords, and into the trachea. Once the patient has been intubated, markings on the ETT can be used as references for how deep the ETT is inserted into the patient's trachea. Once, the desired insertion depth is reached, tape and/or a specialized ETT holder can be used to secure the ETT at the lip of the patient based on a depth of insertion indicated by a centimeter mark on the ETT located closets to the patient's lip. In other words, a centimeter mark on the ETT aligns with the patient's lip level to delineate an insertion depth that the ETT is exhibiting within the patient's trachea. As used herein, the term “lip to tip (“L2T”) length” can refer to a distance from the tip of the ETT positioned within a patient's trachea to the outer surface of a patient's lip (e.g., a top lip or a bottom lip). For example, the outer surface can be the top most surface of the patient's lip when the patient's head is lying horizontally on a supporting surface. Thereby, the L2T length can represent an insertion depth of the ETT. In various embodiments, the L2T length can be used to verify that the ETT is inserted to a correct position within the patient's trachea and/or can be used to verify that the ETT has not been accidently moved from said position.

Wherein the patient is an infant, multiple methods exist for determining the proper L2T length for a subject patient, including, for example: utilization of a mathematical formula based on the infant's EGA, utilization of a mathematical formula based on the infant's weight in kilograms, utilization of one or more tables that present information based on the infant's estimated gestation age and/or weight, ETT vocal cord marks (e.g., reference marks used during intubation), and/or utilization of a measurement of the infant's NTL. Amongst the stated determination methods, use of the NTL measurement can be highly reliable and/or accurate, as compared with other methodologies. For example, a patient's estimated gestational age can be inaccurately assessed, a patient's weight may not be known and/or inaccurately estimated, tables can incorporate varying standards of error, and/or vocal cord marks can be difficult to see when the ETT is inserted into the trachea.

Various embodiments described herein can regard a measuring tape that can comprise one or more indicia for measuring a patient's NTL, wherein the one or more indicia can represent an insertion depth of an ETT based on the NTL. For example, one or more indicia located on the measuring tape can correlate a patient's NTL to a proper L2T length of an ETT. Advantageously, a medical practitioner using the measuring tape can determine an insertion depth for the ETT based on the patient's NTL without preforming any mathematical formulas. Additionally, the measuring tape of the various embodiments herein can increase a likelihood of correctly placing a ETT into a patient's trachea at a position where the ETT would be located generally below the first Thoracic Rib (“T1”) and/or above the carina, and not placed within either the patient's right or left bronchus; thereby providing increased reliability for placement of the ETT at the correct level within the patient's trachea, as compared to conventional techniques.

A correlation between a patient's NTL and a proper ETT insertion depth (e.g., L2T length) has been scientifically validated and shown to be highly accurate. For example, ETT insertion depth (e.g., L2T) based on NTL can be patient specific, regardless of the patient's age, weight, and/or sex. For instance, accuracy of ETT insertion depth (e.g., L2T length) based on NTL can be maintained even with regards to patients born during a multi-birth pregnancy (e.g., twins, triplets, etc.).

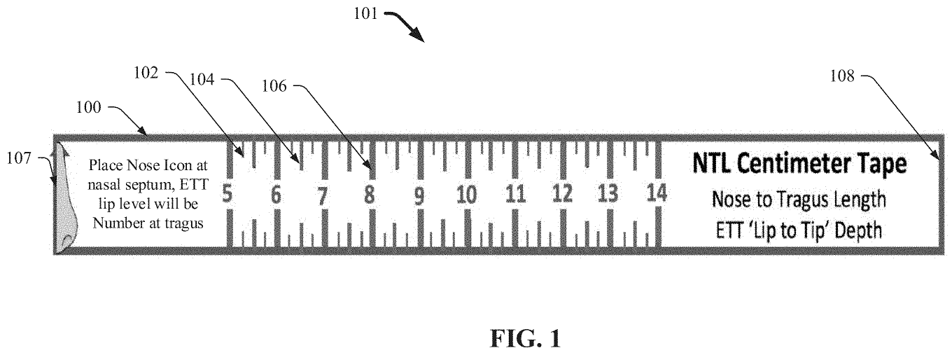

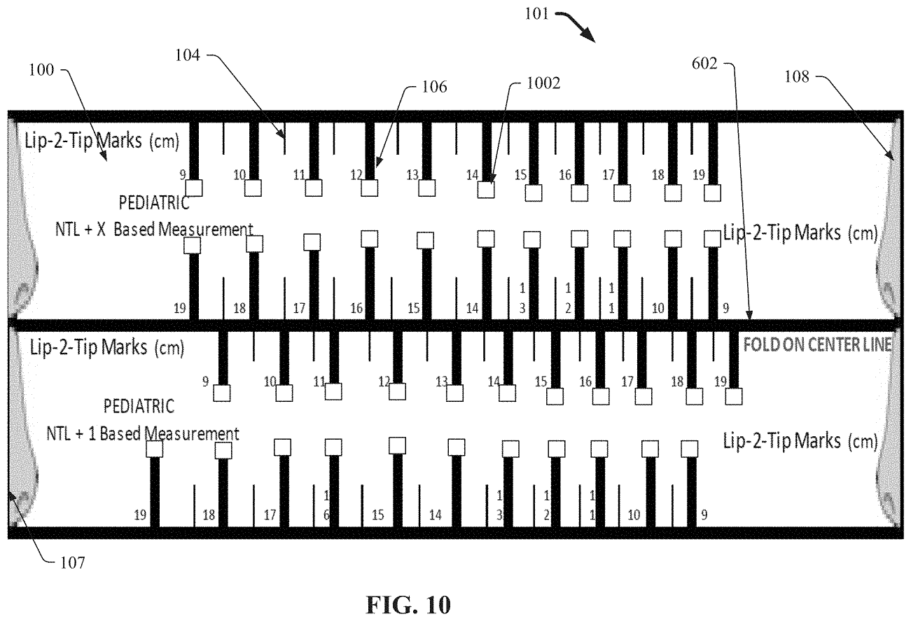

FIG. 1 illustrates a diagram of an example, non-limiting measurement side 100 of an NTL measuring tape 101 in accordance with one or more embodiments described herein. As shown in FIG. 1, the NTL measuring tape 101 can comprise one or more indicia that can directly correlate a patient's NTL to an ETT insertion depth (e.g., a L2T). For example, one or more first indicia 102 can be spaced 0.25 cm apart along the NTL measuring tape 101 to provide increased accuracy with regards to very small infants (e.g., weighing less than 2,500 grams). Additionally, one or more second indicia 104 can comprise one or more marks spaced 0.5 cm apart along the NTL measuring tape 101. Further, one or more third indicia 106 can comprise one or more marks spaced 1 cm apart along the NTL measuring tape 101. The one or more indicia (e.g., one or more first indicia 102, second indicia 104, and/or third indicia 106) can comprise various geometric designs such as lines (e.g., as shown in FIG. 1), circles, square (e.g., as shown in FIG. 10), polygonal shapes, a combination thereof, and/or the like. Additionally, the healthcare provide may make a mark notation within the one or more indicia to better recall the measurement.

The one or more indicia (e.g., the one or more first indicia 102, second indicia 104, and/or third indicia 106) can correlate to an insertion depth for an ETT (e.g., a L2T length). For example, the one or more indicia can correlate an NTL with a L2T length in accordance with Equation 1 below:

Lip to Tip Length=NTL+X (1)

Wherein the lip to tip length can be presented in centimeters, the NTL can be measured in centimeters, and “X” can represent a value greater than or equal to 0.25 cm and less than or equal to 1.5 cm. For example, “X” can represent a value of 1 cm for infants having a weight of greater than 2.5 kg, and/or “X” can represent a value of 0.5 cm for infants having a weight of less than 2.5 kg. For instance, the NTL measuring tape 101 depicted in FIG. 1 can utilize Equation 1, wherein “X” represents a value of 1 cm. The “8” third indicium 106 depicted on the NTL measuring tape 101 shown in FIG. 1 can represent an ETT insertion depth (e.g., a L2T length) of 8 cm based on an NTL of 7 cm, wherein a distance from a first end 107 of the NTL measuring tape 101 to the “8” third indicium 106 can be 7 cm. For instance, the “8” third indicium 106 can be equivalent to a measured NTL of 7 cm.

In one or more embodiments, a medical practitioner using the NTL measuring tape 101 can place the first end 107 of the NTL measuring tape 101 at the patient's nose and extend the NTL measuring tape 101 across the patient's face to the patient's nearest tragus of the patient's ear to correlate the patient's NTL to an ETT insertion depth. For example, the NTL measuring tape 101 can be placed at a patient's nasal septum, and the one or more indicia (e.g., the one or more first indicia 102, second indicia 104, and/or third indicia 106) nearest the patient's tragus can represent the desired insertion depth of an ETT (e.g., a L2T length). For example, when measuring the patient's NTL, if the “8” third indicium 106 presented in the NTL measuring tape 101 shown in FIG. 1 is nearest the patient's tragus, then the ETT insertion depth (e.g., L2T length) can be determined to be 8 cm (e.g., in correlation to the patient's NTL and/or a distance from the first end 107 to the subject third indicium 106). Additionally, in one or more embodiments a medical practitioner can position the first end 107 of the NTL measuring tape 101 at the tragus of the patient's ear and extend the NTL measuring tape 101 to the septum of the patient's nose to derive the ETT insertion depth.

In various embodiments, the NTL measuring tape 101 can be folded horizontally or vertically to allow the measurement side 100 to remain small in size yet provide a larger area for information on the reverse side of the tape. Additionally, in one or more embodiments, the NTL measuring tape 101 can present two or more rows of the one or more indicia. In a first row, the one or more indicia can numerically increase from the first end 107 of the NTL measuring tape 101 to a second end 108 of the NTL measuring tape 101. In a second row, the one or more indicia can numerically increase from the second end 108 to the first end 107. Thereby, a medical provider can easily utilize the NTL measuring tape 101 with the patient's left ear or right ear. Further, wherein the NTL measuring tape 101 can be folded, a first portion of the NTL measuring tape 101 can depict one or more indicia arrangements that correlate to ETT insertion depth based on Equation 1 with a first value for “X”; whereas a second portion of the NTL measuring tape 101 can depict one or more indicia arrangements that correlate to ETT insertion depth based on Equation 1 with a second value for “X”. In various embodiments, the measurement side 100 can be depicted on the front and back side of the NTL measuring tape 101. For example, a front side of the NTL measuring tape 101 can display one or more indicia in accordance with one or more embodiments described herein, and a back side of the NTL measuring tape 101 can display one or more indicia in accordance with one or more embodiments described herein.

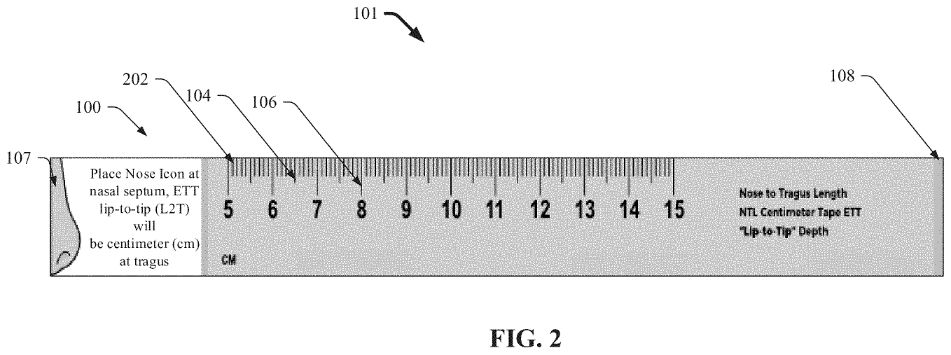

FIG. 2 illustrates a diagram of the example, non-limiting NTL measuring tape 101 comprising one or more fourth indicia 202 in accordance with one or more embodiments described herein. Repetitive description of like elements employed in other embodiments described herein is omitted for sake of brevity. For example, one or more fourth indicia 202 can be spaced 0.1 cm apart along the NTL measuring tape 101 to provide increased accuracy with regards to very small infants (e.g., weighing less than 2,500 grams) and/or mico-premie patients (e.g., weighing less than 1,000 grams). As shown in FIG. 2, one or more embodiments of the NTL measuring tape 101 can comprise the one or more fourth indicia 202 in combination with the one or more second indicia 104, and/or third indicia 106. While FIGS. 1 and 2 depict a plurality of indicia spaced at 0.1 cm, 0.25 cm, 0.50 cm, and/or 1.0 cm intervals, the architecture of the NTL measuring tape 101 is not so limited. For example, embodiments of the NTL measuring tape 101 comprising indicia arranged in one or more other defined intervals are also envisaged and can be incorporated into Equation 1 in accordance with the various embodiments described herein.

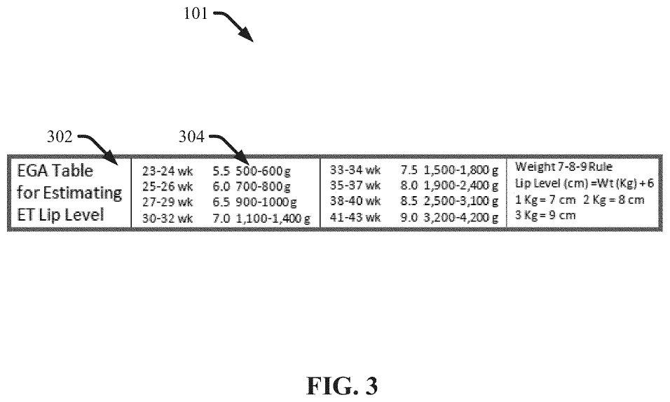

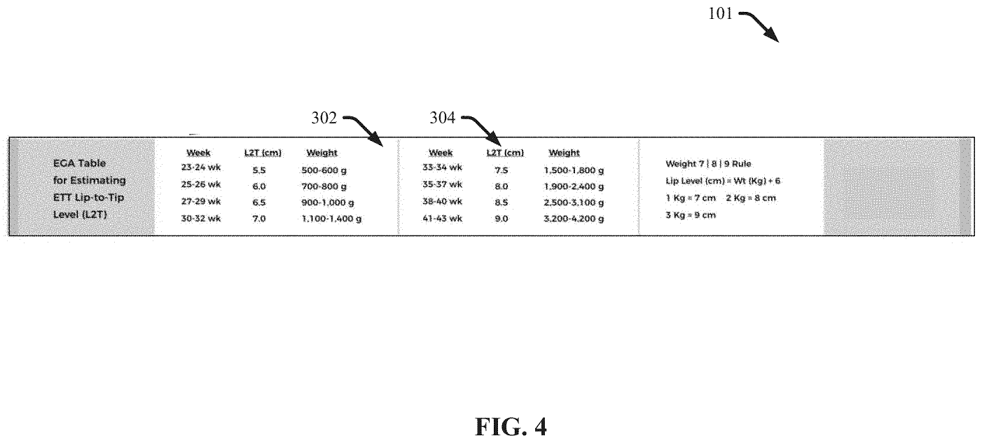

FIGS. 3 and 4 illustrate diagrams of the example, non-limiting NTL measuring tape 101 further comprising a reference side 302 that can present medical information 304 in a readily accessible manner in accordance with one or more of the embodiments described herein. Repetitive description of like elements employed in other embodiments described herein is omitted for sake of brevity. For example, the medical information 304 comprised on the reference side 302 can regard one or more methods for the estimation of ETT insertion depth in addition to the correlation characterized by Equation 1. In addition to referencing the plurality of indicia comprised on the NTL measuring tape 101, a medical provider can reference the medical information 304 to facilitate one or more medical analyses of the patient.

The exemplary embodiments depicted in FIGS. 3 and/or 4, present medical information 304 that includes a table for determining ETT insertion depth (e.g., L2T length) based on EGA and/or weight. In addition, or alternatively, space can be presented for information regarding a subject patient. For example, the reference side 302 of the NTL measuring tape 101 can present areas to fill in: an NTL measuring date, an NTL measurement, a patient name, a birth date of the patient, one or more conditions of the patient's birth, a patient's estimated gestational age, a patient's weight (e.g., in kilograms), a status (e.g., including a date of said status) of the patient, a combination thereof, and/or the like. Thus, a user of the NTL measuring tape 101 can utilize the measurement side 100 (e.g., exemplarily depicted in FIG. 1) to determine an ETT insertion depth (e.g., lip to tip length) based on an NTL and/or can utilize the reference side 302 (e.g., exemplarily depicted in FIGS. 3 and/or 4) to reference additional information (e.g., medical information 304) about the patient and/or ETT insertion techniques.

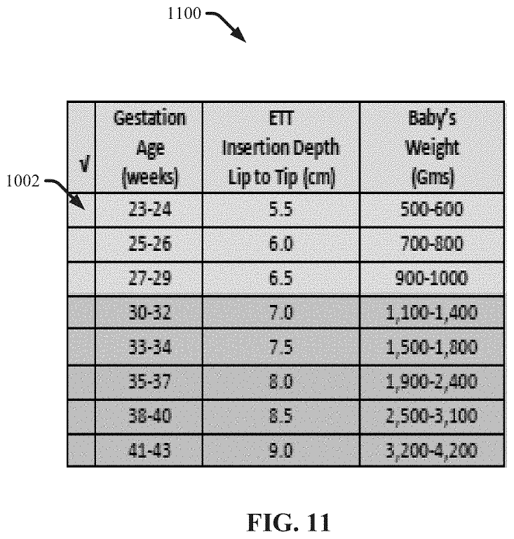

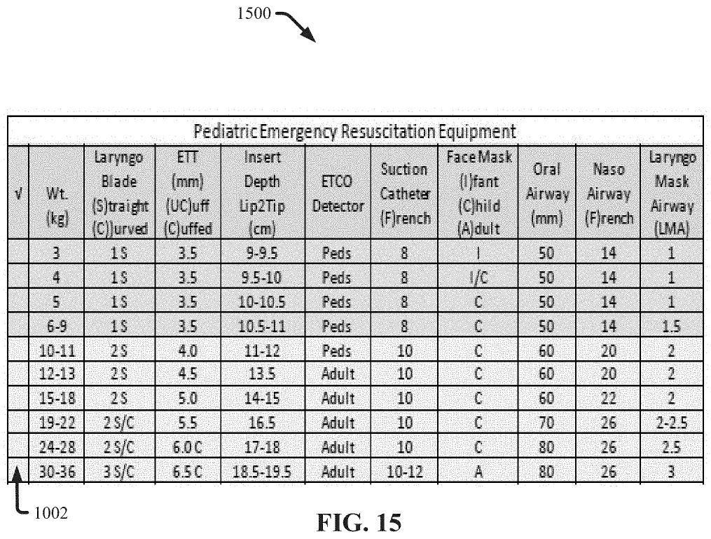

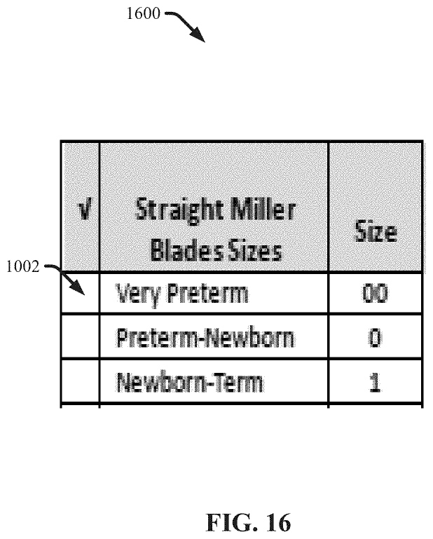

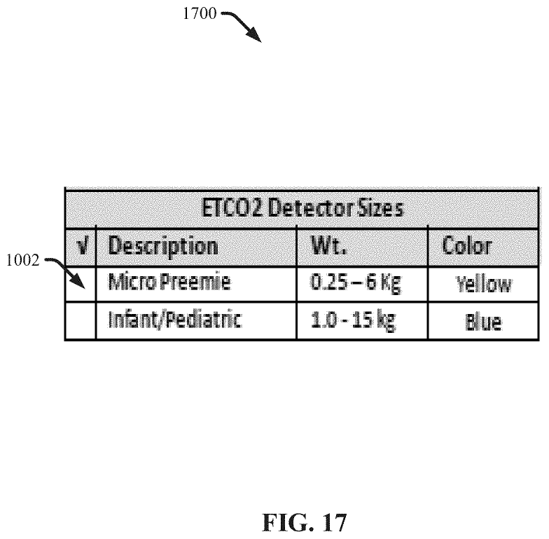

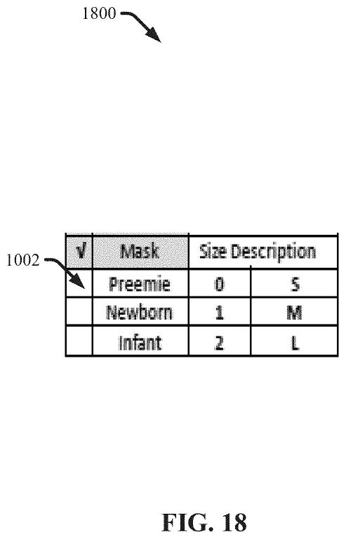

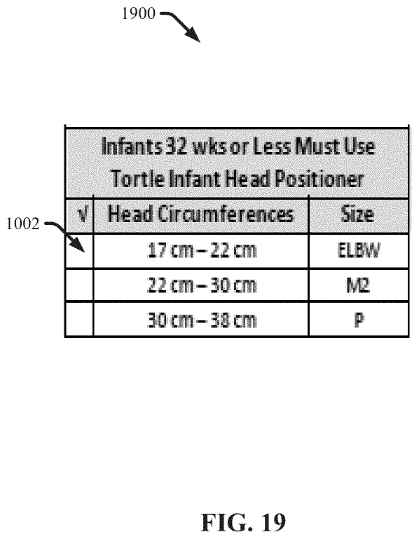

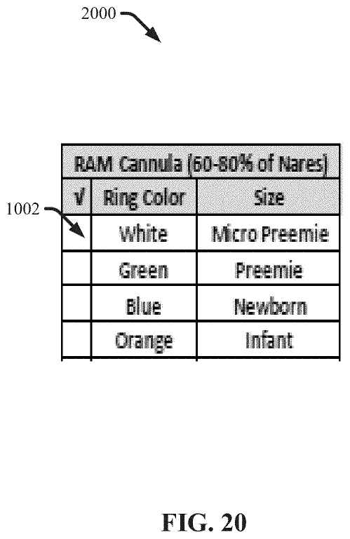

Although FIGS. 3 and 4 depict medical information 304 for estimation of ETT insertion depth (e.g., L2T length) based on EGA-age-based tables, the architecture of the medical information 304 that can be included on the reference side 302 is not so limited. For example, the medical information 304 can further comprise alternate reference formulas, charts, and/or methods for estimating the ETT insertion depth (e.g., such as the medical information depicted in FIGS. 11-20). Additionally, the medical information 304 can regard equipment and/or supplies used in medical emergencies (e.g., respiratory emergencies). Further, in various embodiments the NTL measuring tape 101 can comprise the medical information 304 located on the measurement side 100 in addition to, or alternatively to, the reference side 302. For example, the NTL measuring tape 101 can comprise two measurement sides 100 (e.g., instead of one measurement side 100 and one reference side 302), wherein one or more of the measurement sides 100 comprise reference information (e.g., medical information 304).

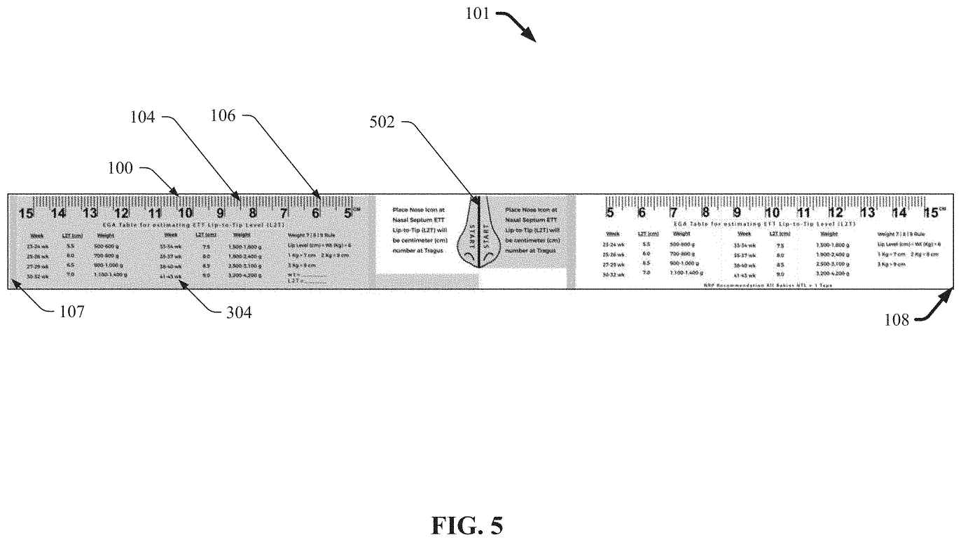

FIG. 5 illustrates a diagram of the example, non-limiting NTL measuring tape 101 comprising a measurement side 100 that can be utilized to measure a patient's NTL and determine the ETT insertion depth using either the left side or the right side of the patient's face in accordance with one or more of the embodiments described herein. Repetitive description of like elements employed in other embodiments described herein is omitted for sake of brevity. As shown in FIG. 5, the NTL measuring tape 101 can comprise a starting point 502 (e.g., delineated by a solid line in FIG. 5) that can serve as the first end 107 or second end 108 of the NTL measuring tape 101. In one or more embodiments, the starting point 502 can be positioned in a center region of the NTL measuring tape 101 (e.g., as depicted in FIG. 5).

For example, the starting point 502 can be placed at the nose of the patient, whereupon a first end 107 can extend across the patient's face towards the patient's tragus. A first portion of the NTL measuring tape 101 between the starting point 502 and the first end 107 can comprise a plurality of indicia in accordance with the various embodiments described herein. An indicia comprised within the first portion of the NTL measuring tape 101 and nearest the patient's tragus can indicate the ETT insertion depth. Additionally, or alternatively, the starting point 502 can be placed at the nose of the patient, whereupon the second end 108 can extend across another side of the patient's face towards the patient's other tragus. The NTL measuring tape 101 can comprise a second portion between the starting point 502 and the second end 108. The second portion can comprise another plurality of indicia in accordance with the various embodiments described herein. An indicia comprised within the second portion of the NTL measuring tape 101 and nearest the patient's other tragus can indicate the ETT insertion depth. Thereby, the starting point 502 can be positioned at the patient's nose, and the NTL measuring tape 101 can extend to either the right or left tragus of the patient to determine the ETT insertion depth.

In various embodiments, the starting point 502 can delineate a location at which a medical provider can fold the NTL measuring tape 101 to create first end 107 or second end 108 of the NTL measuring tape 101. For example, a medical provider can fold the NTL measuring tape 101 along the starting point 502 to create a first end 107 of the NTL measuring tape 101 such that the first end 107 created by the fold can be positioned at the nasal septum of the patient to start measurement of the NTL and/or determination of the ETT insertion depth. By folding the NTL measuring tape 101, the medical provider can readily form embodiments in which the indicia arrangement scales in a preferred direction to facilitate measurement of a desired side of the patient's face.

For example, to the right or left of the starting point 502, the one or more indicia can numerically increase from the starting point 502 to a distal end to measure the NTL. Thereby, a medical provider can fold the NTL measuring tape 101 along the starting point 502 to readily utilize the patient's left ear or right ear. Various embodiments of the NTL measuring tape 101 can depict different icons than the nose icon (e.g., a single line or an ear icon) depicted in the figures. Additionally, FIG. 5 illustrates that the measurement side 100 of the NTL measuring tape 101 can also present medical information 304 for ease of accessibility.

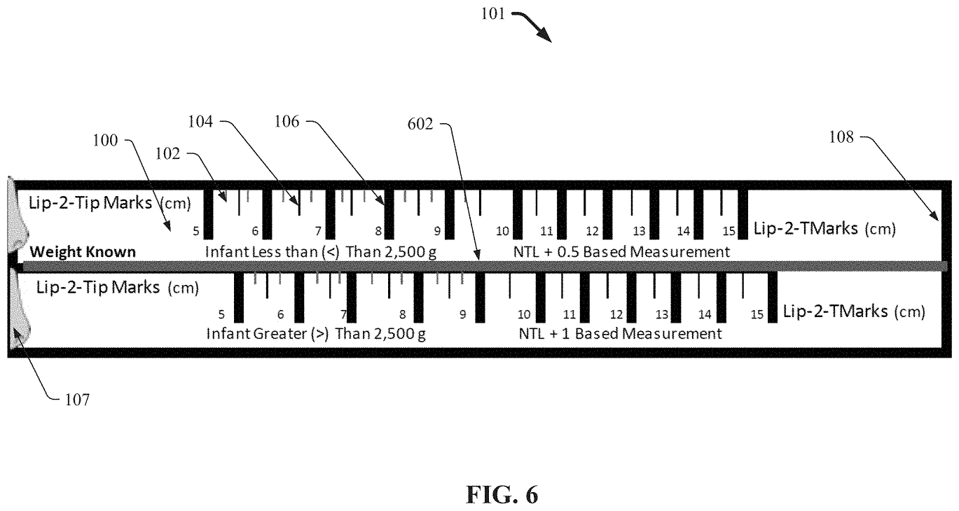

FIG. 6 illustrates a diagram of the example, non-limiting NTL measuring tape 101 further comprising a horizontal folding line 602 in accordance with one or more embodiments described herein. Repetitive description of like elements employed in other embodiments described herein is omitted for sake of brevity. In various embodiments, the NTL measuring tape 101 can be folded horizontally to allow the measurement side 100 to remain small in size yet provide a larger area for medical information 304 on the reference side 302 of the NTL measuring tape 101. For example, once folded along a horizontal folding line 602, the NTL measuring tape 101 can include four surfaces (e.g., two measurement surfaces 100, formed from the top and bottom portions of the NTL measuring tape 101, and two inside surfaces, positioned inside the folded structure of the NTL measuring tape 101).

Additionally, in one or more embodiments the NTL measuring tape 101 can comprise two or more indicia arrangements, wherein the indicia arrangements can be separated by the horizontal folding line 602. For example, a first indicia arrangement can depict one or more indicia that can correlate to ETT insertion depth based on Equation 1 with a first value for “X”; whereas a second indicia arrangement can depict one or more indicia that can correlate to ETT insertion depth based on Equation 1 with a second value for “X”.

As shown in FIG. 6, the horizontal folding line 602 can separate a top portion of the NTL measuring tape 101 and/or a bottom portion of the NTL measuring tape 101. Each of the top portion and the bottom portion can respectively comprise the features described herein. For example, each portion can comprise one or more indicia located between a first end 107 and a second end 108, wherein the one or more indicia can correlate a patient's NTL to an insertion depth for an ETT based on Equation 1. However, respective portions can comprise respective indicia arrangements that can utilize different values for “X” in Equation 1. For example, the top portion shown in FIG. 6 can determine an ETT insertion depth based on Equation 1, wherein “X” has a value of 0.5 cm. In contrast, the bottom portion shown in FIG. 6 can determine an ETT insertion depth based on Equation 1, wherein “X” has a value of 1 cm.

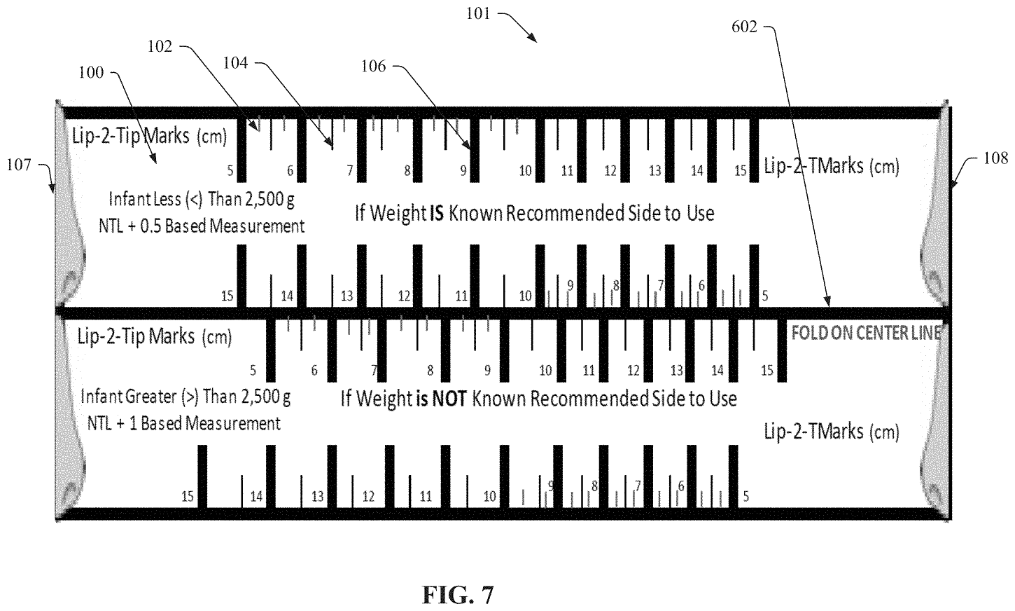

A medical provider can fold the NTL measuring tape 101 depicted in FIG. 6 along the horizontal folding line 602 and choose which portion to utilize for determining the ETT insertion depth. For example, a medical provider can utilize the top portion (e.g., comprising the first indicia arrangement) for patients weighing less than 2.5 kg and the bottom portion (e.g., comprising the second indicia arrangement) for patients weighing more than 2.5 kg. In another example, a medical provider can utilize the top portion when the patient's weight is known and the bottom portion when the patient's weight is unknown. Further, in one or more embodiments the NTL measuring tape 101 can be folded into a number of portions greater than the two portions shown in FIG. 6 (e.g., three or more portions). For instance, the NTL measuring tape 101 can comprise a plurality of horizontal folding lines 602 and/or starting points 502. FIG. 7 illustrates another diagram of the example, non-limiting NTL measuring tape 101 comprising the one or more horizontal folding lines 602 in accordance with one or more embodiments described herein. Repetitive description of like elements employed in other embodiments described herein is omitted for sake of brevity. As shown in FIG. 7, the one or more horizontal folding lines 602 can separate one or more portions of the NTL measuring tape 101, wherein each portion can comprise one or more of the indicia arrangement schemes described herein. For example, the horizontal folding line 602 can delineate a first portion of the NTL measuring tape 101 that comprises two rows of indicia, as shown in FIG. 7.

In one or more embodiments, one or more of the portions of the NTL measuring tape 101 can comprise two rows of indicia numerically increasing in opposite directions (e.g., as shown in FIG. 7) to facilitate use with a patient's left or right ear as described herein. In a first row of the first portion, the one or more indicia can numerically increase from the first end 107 of the NTL measuring tape 101 to the second end 108 of the NTL measuring tape 101. In a second row of the first portion, the one or more indicia can numerically increase from the second end 108 to the first end 107. Thereby, a medical provider can easily utilize the first portion of the NTL measuring tape 101 with the patient's left ear or right ear. Further, the second portion of the NTL measuring tape 101 delineated by the horizontal folding line 602 can also share the same indicia arrangement as the first portion (e.g., as shown in FIG. 7) or can comprise an alternate indicia arrangement.

Moreover, the portions defined by the horizontal folding line 602 can comprise indicia positioned on the NTL measuring tape 101 based on Equation 1. However, respective portions can utilize different values for “X” in Equation 1. For example, the top portion shown in FIG. 7 (e.g., including the two rows of indicia scaling in opposite directions) can determine an ETT insertion depth based on Equation 1, wherein “X” has a value of 0.5 cm. In contrast, the bottom portion shown in FIG. 7 (e.g., including the two rows of indicia scaling in opposite directions) can determine an ETT insertion depth based on Equation 1, wherein “X” has a value of 1 cm.

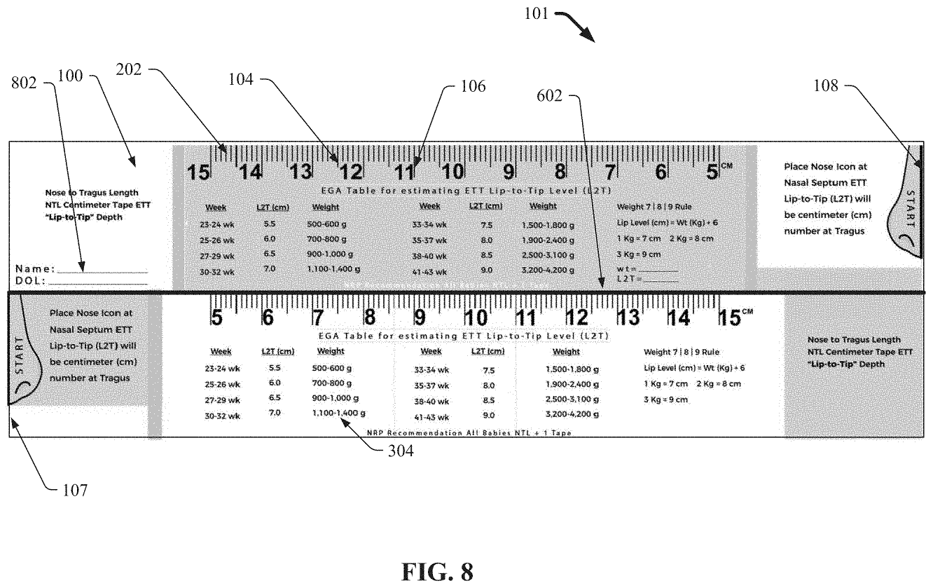

FIG. 8 illustrates a diagram of the example, non-limiting NTL measuring tape 101 comprising a measurement side 100 that can be folded along a horizontal folding line 602 in accordance with one or more embodiments described herein. Repetitive description of like elements employed in other embodiments described herein is omitted for sake of brevity. As shown in FIG. 8, the horizontal folding line 602 can delineate two or more portions of the NTL measuring tape 101, wherein each portion can present a respective indicia arrangement. For instance, a top portion of the NTL measuring tape 101 depicted in FIG. 8 can have an indicia arrangement in which the plurality of indicia increase in value from the second end 108 to the first end 107. In contrast, the bottom portion of the NTL measuring tape 101 depicted in FIG. 8 can have an indicia arrangement in which the plurality of indicia decrease in value from the second end 108 to the first end 107. The varying indicia arrangements between the portions can facilitate use of the NTL measuring tape 101 in multiple configurations (e.g., using the patient's left ear versus right ear as a measuring reference).

As shown in FIG. 8, the measurement side 100 can also comprise medical information 304, such as: alternative reference formula, charts and methods for estimation of L2T length. Further, one or more areas on the measurement side 100 can be reserved to present additional information, such as patient information 802. For example, one or more areas of the measurement side 100 can present: an NTL measuring date, an NTL measurement, a patient name, a birth date of the patient, one or more conditions of the patient's birth, a patient's EGA, a patient's weight (e.g., in kilograms), a status (e.g., including a date of said status) of the patient, a combination thereof, and/or the like. Thus, a user of the NTL measuring tape 101 can utilize the measurement side 100 (e.g., exemplarily depicted in FIG. 8) to determine an ETT insertion depth (e.g., lip to tip length) based on an NTL and/or can utilize the bottom part (e.g., exemplarily depicted in FIG. 8) to reference additional information about the patient and/or ETT insertion techniques. The measurement side 100 can also depict information regarding feature equipment and/or supplies for respiratory emergencies information.

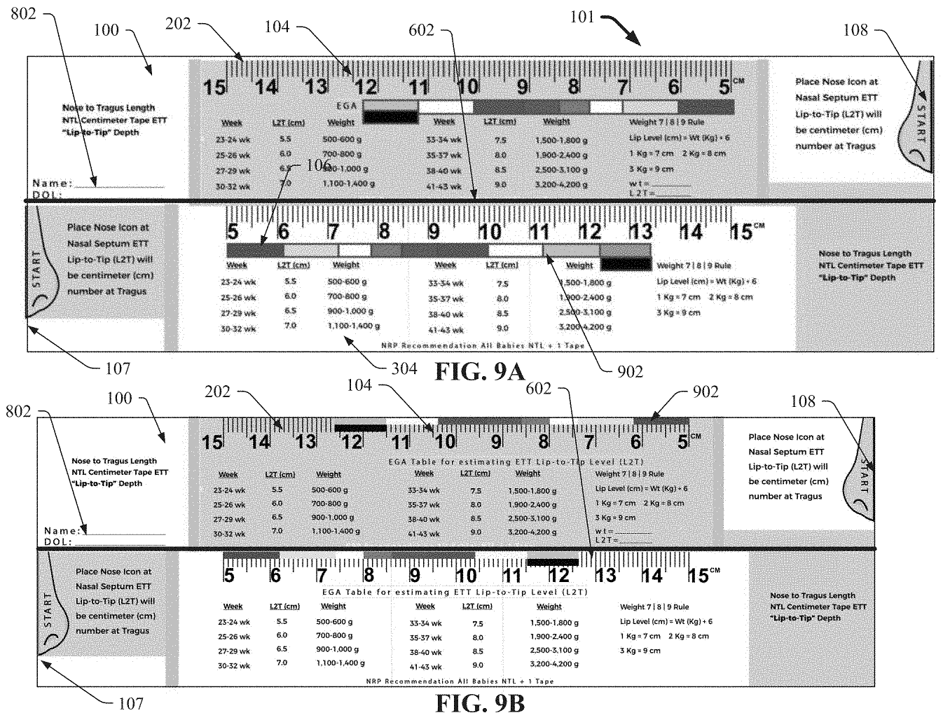

FIGS. 9A and 9B illustrate diagrams of the example, non-limiting NTL measuring tape 101 comprising one or more coding schemes 902 to facilitate identification equipment associated with insertion of an ETT in accordance with one or more embodiments described herein. Repetitive description of like elements employed in other embodiments described herein is omitted for sake of brevity. For example, the coding scheme can comprise color-code bars for selection of appropriate size securing device to use for the patient. As shown in FIGS. 9A and/or 9B, the one or more coding schemes 902 can be utilized in association with the plurality of indicia to facilitate identification of a medical equipment size that can properly fit the given patient. For instance, each color comprised within the color-code bars depicted in FIGS. 9A and/or 9B can be associated with a different size of a given piece of medical equipment (e.g., an ETT securing device).

FIG. 10 illustrates another diagram of the example, non-limiting NTL measuring tape 101 further comprising one or more notation areas 1002 in accordance with one or more embodiments described herein. Repetitive description of like elements employed in other embodiments described herein is omitted for sake of brevity. As shown in FIG. 10, the measurement side 100 of the NTL measuring tape 101 of the various embodiments described herein (e.g., described with regards to FIGS. 1-3) can further comprise one or more notation areas 1002.

For example, the one or more notation areas 1002 can be one or more check-off boxes (e.g., as shown in FIG. 10). A medical provider using the NTL measuring tape 101 can mark the one or more notation areas 1002 (e.g., mark one or more of the check-off boxes) nearest the patient's tragus to make a record of the patients NTL to ETT insertion depth correlation. The one or more notation areas 1002 can be located at one or more distal ends of the one or more indicia. Also, the one or more notation areas 1002 can be located at the distal end of each indicia or selective indicia. For example, the one or more notation areas 402 can be located at the distal end of one or more third indicia 106.

Further, as shown in FIG. 10, one or more embodiments of the NTL measuring tape 101 can be utilized to treat pediatric patients in addition to premature, neonatal, and/or infant patients. For example, the NTL measuring tape 101 for pediatric patients can be longer than other embodiments of the NTL measuring tape 101 to facilitate determinations of large ETT insertion depths. For instance, the pediatric NTL measuring tape 101 shown in FIG. 10 can facilitate determinations of ETT insertion depths less than or equal to 19 cm. Additionally, one or ordinary skill in the art will readily recognize that the notation areas 1002 depicted in FIG. 10 can be included on any of the various NTL measuring tape 101 embodiments described herein.

The NTL measuring tape 101 can be made from a strip of flexible material. Example materials that can comprise the NTL measuring tape 101 can include, but are not limited to: paper, plastic, rubber, metal, a polymer, a combination thereof, and/or the like. Additionally, while 74 indicia (e.g., a plurality of first indicia 102, a plurality of second indicia 104, and/or a plurality of third indicia 106) are shown in FIG. 1, the architecture of the NTL measuring tape 101 is not so limited. For example, embodiments comprising less than or greater than 74 indicia are also envisaged. For instance, the NTL measuring tape 101 can correlate an NTL measurement to an ETT insertion depth greater than or equal to 4 cm and less than or equal to 25 cm. Further, one of ordinary skill in the art will recognize that a variety of designs (e.g., fonts and/or font sizes) can be utilized to present the one or more indicia. Also, one or more instructions can be presented on the NTL measuring tape 101 to facilitate use of the NTL measuring tape 101.

Moreover, in one or more embodiments the NTL measuring tape 101 can be mounted and/or housed within an enclosure (not shown). For example, the NTL measuring tape 101 can be mounted within the enclosure to facilitate extension and/or retraction. For instance, the NTL measuring tape 101 can be mounted such that the tape can extend from the enclosure to facilitate measuring the patient's NTL and can retract into the enclosure to facilitate storage of the NTL measuring tape 101.

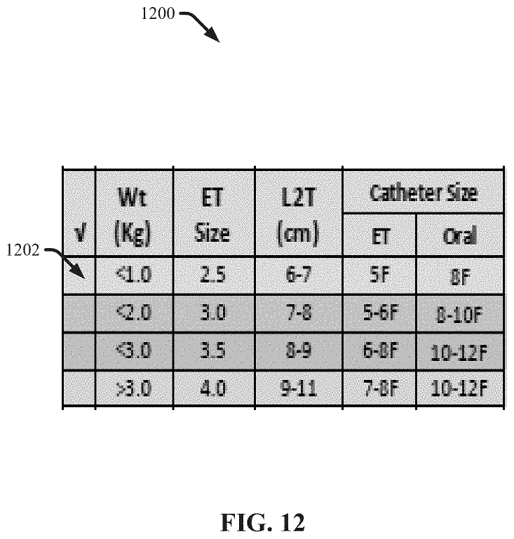

FIGS. 11 and 12 illustrate diagrams of example, non-limiting reference tables 1100 and 1200 that can be included on one or more sides (e.g., measurement side 100 and/or reference side 302) of the NTL measuring tape 101 in accordance with one or more embodiments described herein. Repetitive description of like elements employed in other embodiments described herein is omitted for sake of brevity. Reference table 1100 can provide recommended ETT insertion depths for patients of various gestation age and/or weight. Reference table 1000 can provide recommended ETT insertion depths, ETT sizes, and/or catheter sizes for patients of various weights. Reference table 1200 can provide recommended ETT insertion depths (e.g., L2T lengths) and/or medical equipment sizes based on the patient's weight.

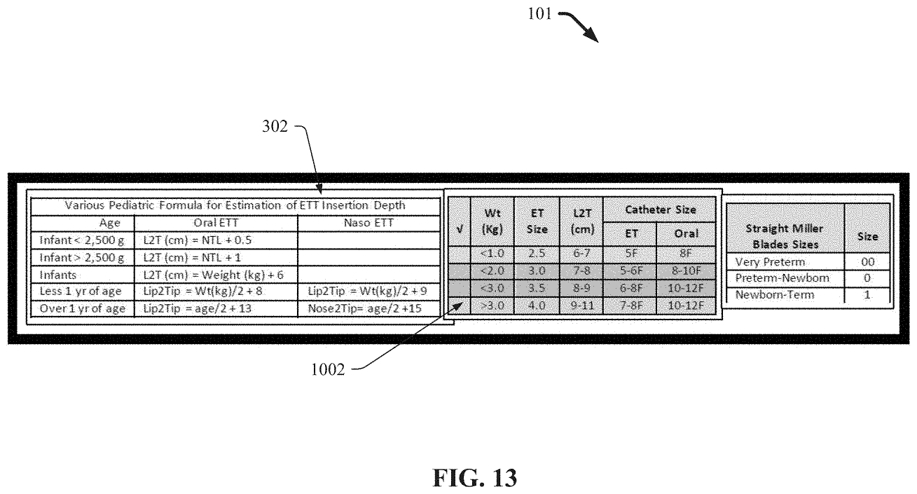

FIG. 13 illustrates another example, non-limiting reference side 302 of the NTL measuring tape 101 in accordance with one or more embodiments described herein. Repetitive description of like elements employed in other embodiments described herein is omitted for sake of brevity. The reference side 302 depicted in FIG. 13 can also depict additional reference information regarding a subject patient and/or an ETT insertion depth (e.g., a L2T length).

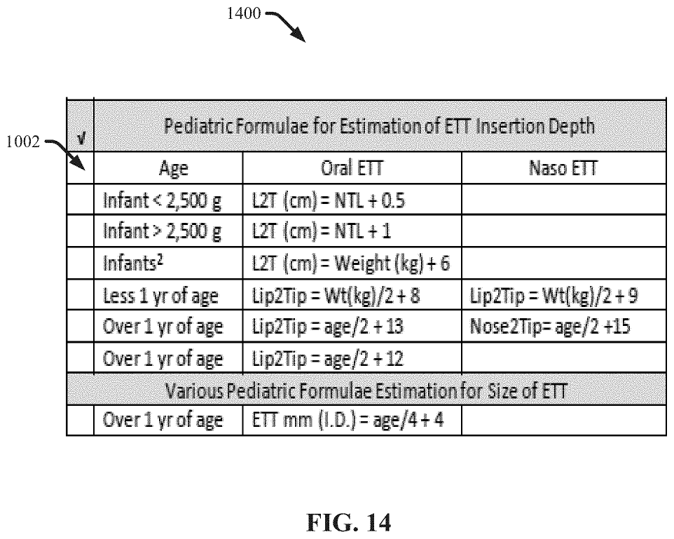

FIGS. 14-20 illustrate diagrams of example, non-limiting reference tables 1400, 1500, 1600, 1700, 1800, 1900, and/or 2000 that can be included on one or more sides (e.g., the measurement side 100 and/or reference side 302) of the NTL measuring tape 101 in accordance with one or more embodiments described herein. Repetitive description of like elements employed in other embodiments described herein is omitted for sake of brevity. In one or more embodiments, the reference tables 1400, 1500, 1600, 1700, 1900, 1900, and/or 2000 can include notation areas 1002 for one or more medical providers to note specific values and/or characteristics depicted on the respective tables than can be applicable to a subject patient.

Reference table 1400 can regard multiple formulae for determining the ETT insertion depth of pediatric patients of various age, weight, and/or size. Reference table 1500 can provide information for various pediatric emergency resuscitation equipment based on a patient's weight. Reference table 1600 can provide multiple straight miller blade sizes for patients of various ages. Reference table 1700 can provide recommended end-tidal carbon dioxide (“ETCO2”) detector sizes for patients of various weights. Reference table 1800 can provide recommended mask sizes for patients of various ages. Reference table 1900 can provide recommended tortle infant head positioner sizes for patients with various head sizes. Reference table 2000 can provide recommended nasal cannula sizes for patients of various ages.

As shown in FIGS. 11-20, the one or more reference tables 1100, 1200, 1400, 1500, 1600, 1700, 1800, 1900, and/or 2000 can present reference medical information and/or patient information. The reference information can regard, for example: information about a subject patient, information about one or more ETT insertion techniques, information to facilitate ETT insertion depth (e.g., L2T length) via one or more methodologies, information regarding ETT insertion equipment, a combination thereof, and/or the like. One of ordinary skill in the art will recognize that one or more (e.g., all) of the reference tables 1100, 1200, 1400, 1500, 1600, 1700, 1800, 1900, and/or 2000, shown in FIGS. 11-20 can be included on the NTL measuring tape 101.

Additionally, the reference information presented on the NTL measuring tape 101 can be directed to numerous categories of patients. For example, while the reference tables 1100, 1200, 1400, 1500, 1600, 1700, 1800, 1900, and/or 2000 shown in shown in FIGS. 11-20 are primarily directed towards neonatal and pediatric patients, various embodiments of the NTL measuring tape 101 that comprise reference information directed towards other types of patients are also envisaged.

Further, one of ordinary skill in the art will recognize that the reference information presented in FIGS. 11-20 is exemplary, and additional or alternative reference information regarding ETT insertion techniques and/or methodology is also envisaged. For example, the reference information described herein can regard equipment needed for endotracheal intubation, which can include, but is not limited to: oxygen flowmeters and/or tubing, suction apparatuses, flexible suction catheters, yankauer tips, manual resuscitation bags and/or masks, oropharyngeal airways, larynoscopes with assorted blades, ETT sizes, tongue depressors, stylets, stethoscopes, tape, syringes, lubricating jellies, magill forceps, local anesthetics, towels, barrier precautions (e.g., gloves, gowns, masks, and/or eyewear), a combination thereof, and/or the like.

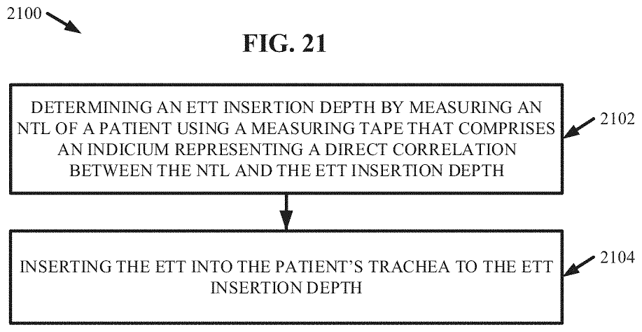

FIG. 21 illustrates a flow diagram of an example, non-limiting method 2100 that can facilitate ETT intubations using an NTL measuring tape 101 in accordance with one or more embodiments described herein. Repetitive description of like elements employed in other embodiments described herein is omitted for sake of brevity.

At 2102, the method 2100 can comprise determining an ETT insertion depth (e.g., a L2T length) by measuring an NTL of a patient using a measuring tape (e.g., NTL measuring tape 101) that can comprise one or more indicia representing a direct correlation between the NTL and the ETT insertion depth. For example, the NTL measuring tape 101 can comprise one or more indicia (e.g., one or more first indicia 102, one or more second indicia 104, one or more third indicia 106, and/or one or more fourth indicia 202) that can represent an ETT insertion depth (e.g., a L2T length) based on the NTL measurement. For instance, the one or more indicia located on the NTL measuring tape 101 can represent a correlation between the NTL length and an ETT insertion depth in accordance with Equation 1 described herein. For example, the NTL measurement tape 101 can comprise one or more first indicia 102 separated from each other at distance of 0.25 cm to provide enhanced precision for determining the ETT insertion depth for extremely low birth weight (“ELBW”) and/or very low birth weight (“VLBW”) infants.

For instance, the determining at 2102 can be facilitated by placing a first end 107 of the NTL measuring tape 101 at the patient's nose (e.g., at a patient's nasal septum) and extending the NTL measuring tape 101 across the patient's face to the patient's nearest ear tragus to measure the patient's NTL. Further, the determining at 2102 can be facilitated by determining the indicium that most closely aligns with the patient's ear tragus during the measuring of the NTL. In one or more embodiments, the NTL measuring tape 101 can comprise one or more notation areas 1002 for recording the determined ETT insertion depth (e.g., for marking the indicium located closest to the patient's ear tragus).

In another instance, the determining at 2102 can be facilitated by placing a first end 107 of the NTL measuring tape 101 at the patient's ear tragus and extending the NTL measuring tape 101 across the patient's face to the patient's nose (e.g., to the patient's nasal septum) to measure the patient's NTL. Further, the determining at 2102 can be facilitated by determining the indicium that most closely aligns with the patient's nose (e.g., nasal septum) during the measuring of the NTL. In one or more embodiments, the NTL measuring tape 101 can comprise one or more notation areas 1002 for recording the determined ETT insertion depth (e.g., for marking the indicium located closest to the patient's nose).

At 2104, the method 2100 can comprise inserting an ETT into the patient's trachea to the ETT insertion depth determined at 2102. For example, the ETT insertion depth determined at 2102 can correlate to a position between the patient's voice cords and carina. For instance, the ETT insertion depth determined at 2102 can correlate to a position between the patient's second rib and third rib, wherein the ETT is not positioned in either the left or right bronchus. Thus, the inserting at 2104 can be properly and expeditiously conducted via utilization of the NTL measuring tape 101 during the determinations at 2102. Additionally, one or more notation areas 1002 (e.g., check boxes) be utilized by the medical provide to annotate specific values on one or more reference tables provided on the NTL measuring tape 101 (e.g., reference tables 1100, 1200, 1400, 1500, 1600, 1700, 1800, 1900, 2000, and/or the like) that can apply to the specific patient undergoing the current procedure.

Various embodiments of the present invention can be directed to computer processing systems, computer-implemented methods, apparatus and/or computer program products that facilitate the efficient, effective, and autonomous (e.g., without direct human guidance) determination of an ETT insertion depth based on one or more NTL measurements. For example, one or more embodiments described herein can regard computer-implemented methods, systems, and/or computer program products that can perform an image analysis process to determine measure a patient's NTL via one or more images of the patient's face. Further, various embodiments described herein can include correlating the NTL measurement derived from the one or more images to an ETT insertion depth (e.g., in accordance with Equation 1).

The computer processing systems, computer-implemented methods, apparatus and/or computer program products employ hardware and/or software to solve problems that are highly technical in nature (e.g., performing an optically based NTL measurement to facilitate determination of an ETT insertion depth), that are not abstract and cannot be performed as a set of mental acts by a human. For example, an individual cannot measure a patient's NTL through a visual analysis of the patient's face with the accuracy and/or efficiency demonstrated by the various embodiments described herein.

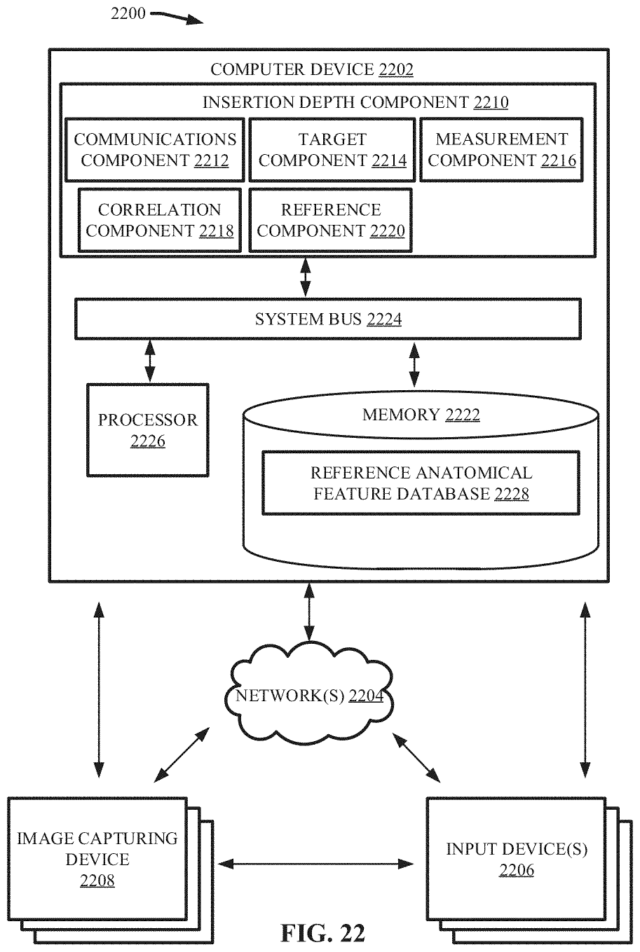

FIG. 22 illustrates a block diagram of an example, non-limiting system 2200 that can determine an ETT insertion depth based on an NTL measurement derived from an image analysis of one or more images depicting the patient's face. Repetitive description of like elements employed in other embodiments described herein is omitted for sake of brevity. Aspects of systems (e.g., system 2200 and the like), apparatuses or processes in various embodiments of the present invention can constitute one or more machine-executable components embodied within one or more machines, e.g., embodied in one or more computer readable mediums (or media) associated with one or more machines. Such components, when executed by the one or more machines, e.g., computers, computing devices, virtual machines, etc. can cause the machines to perform the operations described.

As shown in FIG. 22, the system 2200 can comprise one or more computer devices 2202, one or more networks 2204, input devices 2206, and/or image capturing devices 2208. The computer device 2202 can comprise insertion depth component 2210. The insertion depth component 2210 can further comprise communications component 2212, target component 2214, measurement component 2216, correlation component 2218, and/or reference component 2220. Also, the computer device 2202 can comprise or otherwise be associated with at least one memory 2222. The computer device 2202 can further comprise a system bus 2224 that can couple to various components such as, but not limited to, the insertion depth component 2210 and associated components, memory 2222 and/or a processor 2226. While a computer device 2202 is illustrated in FIG. 22, in other embodiments, multiple devices of various types can be associated with or comprise the features shown in FIG. 22. Further, the computer device 2202 can communicate with one or more cloud computing environments.

The one or more networks 2204 can comprise wired and wireless networks, including, but not limited to, a cellular network, a wide area network (WAN) (e.g., the Internet) or a local area network (LAN). For example, the computer device 2202 can communicate with the one or more input devices 2206 and/or image capturing devices 2208 (and vice versa) using virtually any desired wired or wireless technology including for example, but not limited to: cellular, WAN, wireless fidelity (Wi-Fi), Wi-Max, WLAN, Bluetooth technology, a combination thereof, and/or the like. Further, although in the embodiment shown the insertion depth component 2210 can be provided on the one or more computer devices 2210, it should be appreciated that the architecture of system 2200 is not so limited. For example, the insertion depth component 2210, or one or more components of insertion depth component 2210, can be located at another computer device, such as another server device, a client device, etc.

The one or more input devices 2206 can comprise one or more computerized devices, which can include, but are not limited to: personal computers, desktop computers, laptop computers, cellular telephones (e.g., smart phones), computerized tablets (e.g., comprising a processor), smart watches, keyboards, touch screens, mice, a combination thereof, and/or the like. A user of the system 2200 can utilize the one or more input devices 2206 to input data into the system 2200, thereby sharing (e.g., via a direct connection and/or via the one or more networks 2204) said data with the computer device 2202 and/or image capturing device 2208. For example, the one or more input devices 2206 can send data to the communications component 2212 (e.g., via a direct connection and/or via the one or more networks 2204). Additionally, the one or more input devices 2206 can comprise one or more displays that can present one or more outputs generated by the system 2200 to a user. For example, the one or more displays can include, but are not limited to: cathode tube display (“CRT”), light-emitting diode display (“LED”), electroluminescent display (“ELD”), plasma display panel (“PDP”), liquid crystal display (“LCD”), organic light-emitting diode display (“OLED”), a combination thereof, and/or the like.

A user of the system 2200 can utilize the one or more input devices 2206 and/or the one or more networks 2204 to input one or more settings and/or commands into the system 2200. For example, in the various embodiments described herein, a user of the system 2200 can operate and/or manipulate the computer device 2202 and/or associate components via the one or more input devices 2206. Additionally, a user of the system 2200 can utilize the one or more input devices 2206 to display one or more outputs (e.g., displays, data, visualizations, and/or the like) generated by the computer device 2202, associate components, and/or image capturing device 2208. Further, in one or more embodiments, the one or more input devices 1226 can be comprised within, and/or operably coupled to, a cloud computing environment.

In various embodiments, a user of the system 2200 can input patient information and/or medical technique preferences into the system 2200 via the one or more input devices 2206. For example, the user can utilize the one or more input devices 2206 to enter patient information 802 into the system 2200. Example patient information 802 that can be entered via the one or more input devices 2206 can include, but are not limited to: patient's name, patient's weight, patient's EGA, current date, date of ETT insertion, a combination thereof, and/or the like.

The one or more image capturing devices 2208 can capture image data of a patient's face to facilitate digital measurement of the patient's NTL. For example, the one or more image capturing devices 2208 can comprise one or more cameras and/or camera equipment. The imaging data captured by the one or more image capturing devices 2208 can regard still images of the patient's face and/or video of the patient's face. For instance, the one or more image capturing devices 2208 can capture image data (e.g., photos and/or video) of at least one side of the patient's face, such that the image data depicts the patient's tragus and nose (e.g., nasal septum).

In various embodiments, the one or more computer devices 2202, input devices 2206, and/or image capturing devices 2208 can be comprised within the system 2200 separately and can communicate with each other (e.g., share data) via a direct electrical connection and/or the one or more networks 2204. In one or more embodiments, the one or more computer devices 2202, input devices 2206, and/or image capturing devices 2208 can be comprised within the same structure. For example, the one or more input devices 2206 and/or image capturing devices 2208 can be comprised within the one or more computer devices 2202. For instance, the one or more computer devices 2202 can be a smartphone and/or tablet that incorporates the one or more input devices 2206 (e.g., a touchscreen) and/or image capturing devices 2208 (e.g., camera).

The insertion depth component 2210 can measure a patient's NTL via one or more image analysis processes. For example, the one or more image analysis processes can comprise capturing image data regarding the patient's face (e.g., photos and/or video depicting at least one side of the patient's face, including the patient's tragus and nasal septum). Further, the one or more image analysis processes can include analyzing the image data to identify target reference points comprised within the image data that correlate to the patient's tragus and nasal septum. Additionally, the one or more image analysis processes can comprise measuring the distance between the target references points to determine the patient's NTL. Moreover, the one or more image analysis processes can comprise correlating the determined NTL to an ETT insertion depth (e.g., in accordance with the relationship characterized by Equation 1). Also, the one or more image analysis processes can include presenting reference information (e.g., medical information 304 and/or patient information 802, such as the exemplary reference information depicted in FIGS. 11-20).

The communications component 2212 can receive image data from the one or more image capturing devices 2208 and/or patient information entered by a user via the one or more input devices 2206. Further, the communications component 2212 can share the image data and/or patient information with the associate components of the insertion depth component 2210. Additionally, the communications component 2212 can facilitate communication (e.g., data sharing) between the associate components of the insertion depth component 2210.

The target component 2214 can identify one or more target reference points comprised within image data of a given patient's face captured by the one or more image capturing devices 2208. For example, the target component 2214 can identify a first target reference point that correlates to the location of the patient's tragus, as delineated in the image data. Further, the target component 2214 can identify a second target reference point that correlates to the location of the patient's nasal septum, as delineated in the image data. In various embodiments, the target component 2214 can identify the one or more target reference points based on structural features associated with the patient's anatomy.

For instance, the target component 2214 can identify the first target reference point by analyzing anatomical features of the patient's face delineated by the image data and associating one or more of the anatomical features with the patient's tragus. Thereby, the location of the anatomical features characterizing the patient's tragus can be the location of the first target reference point and/or the location of the patient's tragus. For example, reference anatomical features characterizing the general shape of a tragus can be stored in memory 2222, and the target component 2214 can compare anatomical features comprised within the image data to the reference anatomical features to determine whether the given anatomical features correlate to the patient's tragus.

In another instance, the target component 2214 can identify the second target reference point by analyzing anatomical features of the patient's face delineated by the image data and associate one or more of the anatomical features with the patient's nose (e.g., nasal septum). Thereby, the location of the anatomical features characterizing the patient's nose can be the location of the second target reference point, and/or the location of the patient's nose. For example, reference anatomical features characterizing the general shape of a nose (e.g., nasal septum) can be stored in memory 2222, and the target component 2214 can compare anatomical features comprised within the image data to the reference anatomical features to determine whether the given anatomical features correlate to the patient's nose (e.g., nasal septum).

In one or more embodiments, the target component 2214 can utilize one or more artificial intelligence (“AI”) technologies, such as machine learning, to identify the one or more target reference points. As used herein, the term “machine learning” can refer to an application of AI technologies to automatically and/or autonomously learn and/or improve from an experience (e.g., training data) without explicit programming of the lesson learned and/or improved. For example, machine learning tasks can utilize one or more algorithms to facilitate supervised and/or unsupervised learning to perform tasks such as classification, regression, and/or clustering. In various embodiments, the target component 2214 can utilize machine learning to create and/or update reference anatomical features (e.g., stored in memory 2222) that can be used in comparison of the captured image data to identify the one or more target reference points.

For example, in one or more embodiments the target component 2214 can generate one or more neural network models to generate, update, and/or maintain a reference anatomical feature database 2228 that can comprise the reference anatomical features. As used herein, the term “neural network model” can refer to a computer model that can be used to facilitate one or more machine learning tasks, wherein the computer model can simulate a number of interconnected processing units that can resemble abstract versions of neurons. For example, the processing units can be arranged in a plurality of layers (e.g., one or more input layers, one or more hidden layers, and/or one or more output layers) connected with by varying connection strengths (e.g., which can be commonly referred to within the art as “weights”). Neural network models can learn through training, wherein data with known outcomes is inputted into the computer model, outputs regarding the data are compared to the known outcomes, and/or the weights of the computer model are autonomous adjusted based on the comparison to replicate the known outcomes. As used herein, the term “training data” can refer to data and/or data sets used to train one or more neural network models. As a neural network model trains (e.g., utilizes more training data), the computer model can become increasingly accurate; thus, trained neural network models can accurately analyze data with unknown outcomes, based on lessons learning from training data, to facilitate one or more machine learning tasks. Example neural network models can include, but are not limited to: perceptron (“P”), feed forward (“FF”), radial basis network (“RBF”), deep feed forward (“DFF”), recurrent neural network (“RNN”), long/short term memory (“LSTM”), gated recurrent unit (“GRU”), auto encoder (“AE”), variational AE (“VAE”), denoising AE (“DAE”), sparse AE (“SAE”), markov chain (“MC”), Hopfield network (“HN”), Boltzmann machine (“BM”), deep belief network (“DBN”), deep convolutional network (“DCN”), deconvolutional network (“DN”), deep convolutional inverse graphics network (“DCIGN”), generative adversarial network (“GAN”), liquid state machining (“LSM”), extreme learning machine (“ELM”), echo state network (“ESN”), deep residual network (“DRN”), kohonen network (“KN”), support vector machine (“SVM”), and/or neural turing machine (“NTM”).

In one or more embodiments, the target component 2214 can impose one or more target icons (e.g., squares, circles, and/or the like) onto the image data at the location of the one or more target reference points. Further, target component 2214 can share the captured image and/or video with the imposed targets icons with a user of the system 2200 (e.g., via the one or more input devices 2206 and/or networks 2204). For example, the user can view the captured image and/or video of the patient's face with the one or more target icons displayed to delineate the locations of the patient's tragus and/or nose, as identified by the target component 2214. Thereby, the user of the system 2200 can verify that the target component 2214 has correctly identified the location of the patient's tragus and/or nose (e.g., nasal septum) within the image data.

Further, in various embodiments the user can utilize the one or more input devices 2206 to re-position one or more of the target icons on the captured image and/or video. For example, the user can utilize the one or more input devices 2206 (e.g., a touch screen) to move (e.g., drag) one or more of the target icons across the captured image and/or video to correct an identification of the patient's tragus and/or nose (e.g., nasal septum). Additionally, wherein the user re-positions one or more of the target icons, and thereby one or more of the target reference points, the target component 2214 can learn (e.g., via one or more machine learning tasks, such as neural network models) from the location correction and/or update the reference anatomical features comprised within the one or more reference anatomical databases 2228 to increase the accuracy of subsequent identifications. For instances, the target component 2214 can learn from one or more re-positionings to further refine reference anatomical features characterizing a tragus and/or nose.

In one or more embodiments, the measurement component 2216 measure a distance between the identified target reference points based on the image data to determine the patient's NTL. For example, the measurement component 2216 can utilize a digital measuring algorithm to determine the distance between the target reference points and thereby the patient's NTL.

In one or more embodiments, the measurement component 2216 can measure the distance based on meta data (e.g., an exchangeable image file (“EXIF”)) comprised within the captured image data. For instance, the meta data can describe one or more camera features of the image capturing device 2208, such as, for example, the lens size, f-stop, and/or zoom utilized to capture the image data. Further, the one or more image capturing devices 2208 can capture the image data from a defined distance from the patient's face. Based on the defined distance and the meta data of the captured image and/or video, the measurement component 2216 can determine a distance between the identified target reference points.

Also, in one or more embodiments the one or more image capturing devices 2208 can impose one or more boundary lines onto a display used to operate the image capturing devices 2208. For example, positioning of the boundary lines can be based on the one or more camera features of the image capturing device 2208, such that moving the image capturing device 2208 so as to position the patient's head within the boundary lines can inherently move the image capturing device 2208 to the defined distance. In one or more embodiments, the measurement component 2216 can generate a scale relating distance within the image data to the distance between the patient's ear and nose, such that measuring the distance between the target reference points can digitally measure the NTL.

In one or more embodiments, the correlation component 2218 can determine an ETT insertion depth based on the digitally measured NTL (e.g., measured by the measurement component 2216) and Equation 1. For example, the correlation component 2218 can define a value for “X” in Equation 1 based on a user preference and/or patient information entered into the system 2200 via the one or more input devices 2206. For instance, wherein the patient's weight (e.g., as defined by a user using the one or more input devices 2206) is less than 2,500 g, the correlation component 2218 can determine the ETT insertion depth based on the digitally measured NTL and Equation 1 using a value for “X” of 0.5. Alternatively, wherein the patient's weight (e.g., as defined by a user using the one or more input devices 2206) is greater than 2,500 g, the correlation component 2218 can determine the ETT insertion depth based on the digitally measured NTL and Equation 1 using a value for “X” of 1. In another instance, the user of the system 2200 can manually set the value of “X” using the one or more input devices 2206.

In various embodiments, the correlation component 2218 can display the determined ETT insertion depth on the one or more input devices 2206 for review by a user of the system 2200. For example, the correlation component 2218 can display the target icons, NTL measurement, and/or ETT insertion depth superimposed on the captured image and/or video of the patient (e.g., via the one or more input devices 2206).

In one or more embodiments, reference component 2220 can further display reference information, such as medical information 304 and/or patient information 802, for review by the user of the system 2200. For example, the reference information can be displayed via one or more charts, tables, and/or formulas (e.g., as depicted in FIGS. 11-20). Additionally, reference component 2220 can display the reference information alongside the target icons, NTL measurement, and/or ETT insertion depth on the captured image and/or video of the patient (e.g., via the one or more input devices 2206).

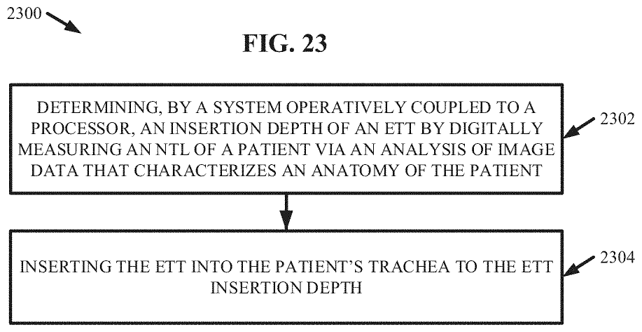

FIG. 23 illustrates a flow diagram of an example, non-limiting method 2300 that can determine an ETT insertion depth based on a digital measurement of a patient's NTL from a captured image of the patient in accordance with one or more embodiments described herein. Repetitive description of like elements employed in other embodiments described herein is omitted for sake of brevity.

At 2302, the method 2300 can comprise determining (e.g., via insertion depth component 2210), by a system 2200 operatively coupled to a processor 2226, an ETT insertion depth by digitally measuring an NTL of a patient via an analysis of image data that characterizes an anatomy of the patient. For example, the determining at 2302 can comprise capturing (e.g., via one or more image capturing devices 2208) the image data (e.g., photos and/or videos) of the patient's face, including the patient's tragus and nose (e.g., nasal septum), in accordance with the various embodiments described herein. Further, the determining at 2302 can comprise identifying (e.g., via target component 2214) one or more target reference points in the image data that can correlate to the location of the patient's tragus and/or nose (e.g., nasal septum) in accordance with the various embodiments described herein. For example, identifying the target reference points can be performed by comparing anatomical features characterized by the image data with reference anatomical features of a tragus and/or nose (e.g., comprised within a reference anatomical feature database 2228) as described herein.

In addition, the determining at 2302 can comprise measuring (e.g., via the measurement component 2216) a distance between the target reference points of the image data via one or more digital measuring algorithms to determine the NTL in accordance with the various embodiments described herein. Moreover, the determining at 2302 can comprise correlating (e.g., via correlation component 2218) the NTL to the ETT insertion depth via one or more relationships characterized by Equation 1. In one or more embodiments, the method 2300 can also comprise displaying the target reference points, NTL digital measurement, and/or ETT (e.g., via one or more input devices 2206) in accordance with the various embodiments described herein. Also, the method 2300 can comprise displaying reference information, such as medical information 304 and/or patient information 802 (e.g., via one or more input devices 2206) in accordance with the various embodiments described herein.

At 2304, the method 2300 can comprise inserting an ETT into the patient's trachea to the ETT insertion depth determined at 2302. For example, the ETT insertion depth determined at 2302 can correlate to a position between the patient's voice cords and carina. For instance, the ETT insertion depth determined at 2302 can correlate to a position between the patient's second rib and third rib, wherein the ETT is not positioned in either the left or right bronchus. Thus, the inserting at 2304 can be properly and expeditiously conducted via digitally measuring captured image data during the determinations at 2302.

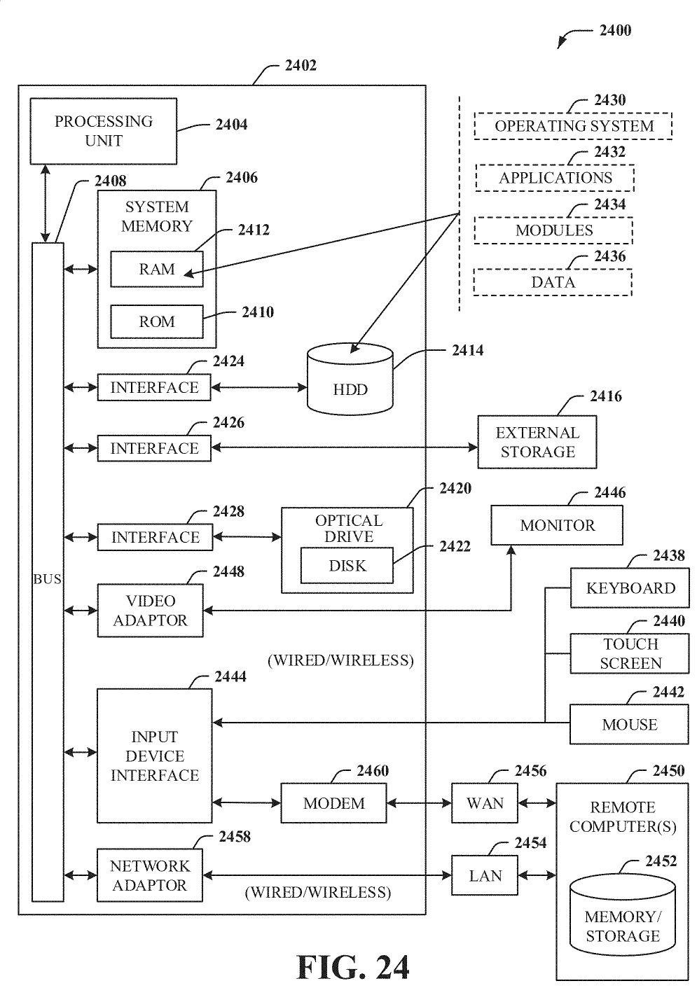

In order to provide additional context for various embodiments described herein, FIG. 24 and the following discussion are intended to provide a brief, general description of a suitable computing environment 2400 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, Internet of Things (“IoT”) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (“RAM”), read only memory (“ROM”), electrically erasable programmable read only memory (“EEPROM”), flash memory or other memory technology, compact disk read only memory (“CD-ROM”), digital versatile disk (“DVD”), Blu-ray disc (“BD”) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 24, the example environment 2400 for implementing various embodiments of the aspects described herein includes a computer 2402, the computer 2402 including a processing unit 2404, a system memory 2406 and a system bus 2408. The system bus 2408 couples system components including, but not limited to, the system memory 2406 to the processing unit 2404. The processing unit 2404 can be any of various commercially available processors. Dual microprocessors and other multi-processor architectures can also be employed as the processing unit 2404.

The system bus 2408 can be any of several types of bus structure that can further interconnect to a memory bus (with or without a memory controller), a peripheral bus, and a local bus using any of a variety of commercially available bus architectures. The system memory 2406 includes ROM 2410 and RAM 2412. A basic input/output system (“BIOS”) can be stored in a non-volatile memory such as ROM, erasable programmable read only memory (“EPROM”), EEPROM, which BIOS contains the basic routines that help to transfer information between elements within the computer 2402, such as during startup. The RAM 2412 can also include a high-speed RAM such as static RAM for caching data.

The computer 2402 further includes an internal hard disk drive (“HDD”) 2414 (e.g., EIDE, SATA), one or more external storage devices 2416 (e.g., a magnetic floppy disk drive (“FDD”) 2416, a memory stick or flash drive reader, a memory card reader, etc.) and an optical disk drive 2420 (e.g., which can read or write from a CD-ROM disc, a DVD, a BD, etc.). While the internal HDD 2414 is illustrated as located within the computer 2402, the internal HDD 2414 can also be configured for external use in a suitable chassis (not shown). Additionally, while not shown in environment 2400, a solid state drive (“SSD”) could be used in addition to, or in place of, an HDD 2414. The HDD 2414, external storage device(s) 2416 and optical disk drive 2420 can be connected to the system bus 2408 by an HDD interface 2424, an external storage interface 2426 and an optical drive interface 2428, respectively. The interface 2424 for external drive implementations can include at least one or both of Universal Serial Bus (“USB”) and Institute of Electrical and Electronics Engineers (“IEEE”) 1394 interface technologies. Other external drive connection technologies are within contemplation of the embodiments described herein.

The drives and their associated computer-readable storage media provide nonvolatile storage of data, data structures, computer-executable instructions, and so forth. For the computer 2402, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 2412, including an operating system 2430, one or more application programs 2432, other program modules 2434 and program data 2436. All or portions of the operating system, applications, modules, and/or data can also be cached in the RAM 2412. The systems and methods described herein can be implemented utilizing various commercially available operating systems or combinations of operating systems.

Computer 2402 can optionally comprise emulation technologies. For example, a hypervisor (not shown) or other intermediary can emulate a hardware environment for operating system 2430, and the emulated hardware can optionally be different from the hardware illustrated in FIG. 24. In such an embodiment, operating system 2430 can comprise one virtual machine (“VM”) of multiple VMs hosted at computer 2402. Furthermore, operating system 2430 can provide runtime environments, such as the Java runtime environment or the .NET framework, for applications 2432. Runtime environments are consistent execution environments that allow applications 2432 to run on any operating system that includes the runtime environment. Similarly, operating system 2430 can support containers, and applications 2432 can be in the form of containers, which are lightweight, standalone, executable packages of software that include, e.g., code, runtime, system tools, system libraries and settings for an application.

Further, computer 2402 can be enable with a security module, such as a trusted processing module (“TPM”). For instance with a TPM, boot components hash next in time boot components, and wait for a match of results to secured values, before loading a next boot component. This process can take place at any layer in the code execution stack of computer 2402, e.g., applied at the application execution level or at the operating system (“OS”) kernel level, thereby enabling security at any level of code execution.

A user can enter commands and information into the computer 2402 through one or more wired/wireless input devices, e.g., a keyboard 2438, a touch screen 2440, and a pointing device, such as a mouse 2442. Other input devices (not shown) can include a microphone, an infrared (“IR”) remote control, a radio frequency (“RF”) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 2404 through an input device interface 2444 that can be coupled to the system bus 2408, but can be connected by other interfaces, such as a parallel port, an IEEE 1394 serial port, a game port, a USB port, an IR interface, a BLUETOOTH® interface, etc.

A monitor 2446 or other type of display device can be also connected to the system bus 2408 via an interface, such as a video adapter 2448. In addition to the monitor 2446, a computer typically includes other peripheral output devices (not shown), such as speakers, printers, etc.

The computer 2402 can operate in a networked environment using logical connections via wired and/or wireless communications to one or more remote computers, such as a remote computer(s) 2450. The remote computer(s) 2450 can be a workstation, a server computer, a router, a personal computer, portable computer, microprocessor-based entertainment appliance, a peer device or other common network node, and typically includes many or all of the elements described relative to the computer 2402, although, for purposes of brevity, only a memory/storage device 2452 is illustrated. The logical connections depicted include wired/wireless connectivity to a local area network (“LAN”) 2454 and/or larger networks, e.g., a wide area network (“WAN”) 2456. Such LAN and WAN networking environments are commonplace in offices and companies, and facilitate enterprise-wide computer networks, such as intranets, all of which can connect to a global communications network, e.g., the Internet.

When used in a LAN networking environment, the computer 2402 can be connected to the local network 2454 through a wired and/or wireless communication network interface or adapter 2458. The adapter 2458 can facilitate wired or wireless communication to the LAN 2454, which can also include a wireless access point (“AP”) disposed thereon for communicating with the adapter 2458 in a wireless mode.

When used in a WAN networking environment, the computer 2402 can include a modem 2460 or can be connected to a communications server on the WAN 2456 via other means for establishing communications over the WAN 2456, such as by way of the Internet. The modem 2460, which can be internal or external and a wired or wireless device, can be connected to the system bus 2408 via the input device interface 2444. In a networked environment, program modules depicted relative to the computer 2402 or portions thereof, can be stored in the remote memory/storage device 2452. It will be appreciated that the network connections shown are example and other means of establishing a communications link between the computers can be used.

When used in either a LAN or WAN networking environment, the computer 2402 can access cloud storage systems or other network-based storage systems in addition to, or in place of, external storage devices 2416 as described above. Generally, a connection between the computer 2402 and a cloud storage system can be established over a LAN 2454 or WAN 2456 e.g., by the adapter 2458 or modem 2460, respectively. Upon connecting the computer 2402 to an associated cloud storage system, the external storage interface 2426 can, with the aid of the adapter 2458 and/or modem 2460, manage storage provided by the cloud storage system as it would other types of external storage. For instance, the external storage interface 2426 can be configured to provide access to cloud storage sources as if those sources were physically connected to the computer 2402.

The computer 2402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (“Wi-Fi”) and BLUETOOTH® wireless technologies. Thus, the communication can be a predefined structure as with a conventional network or simply an ad hoc communication between at least two devices.

What has been described above include mere examples of systems, computer program products and computer-implemented methods. It is, of course, not possible to describe every conceivable combination of components, products and/or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A measuring tape, comprising:
an indicium representing an insertion depth of an endotracheal tube based upon a direct correlation between a nasal to tragus length of a patient and the insertion depth.

2. The measuring tape of claim 1, wherein the indicium is located on a strip of flexible material.

3. The measuring tape of claim 1, wherein the indicium is comprised within a plurality of indicia.

4. The measuring tape of claim 3, wherein the plurality of indicia comprise a first set of indicia and a second set of indicia, wherein the first set of indicia represent the insertion depth for a first category of patient, and wherein the second set of indicia represent the insertion depth for a second category of patient.

5. The measuring tape of claim 4, wherein the first set of indicia are positioned on a first portion of the measuring tape, and wherein the second set of indicia are positioned on a second portion of the measuring tape.

6. The measuring tape of claim 3, wherein adjacent indicia of the plurality of indicia are spaced 1 centimeter apart from each other.

7. The measuring tape of claim 3, wherein adjacent indicia of the plurality of indicia are spaced 0.5 centimeter apart from each other.

8. The measuring tape of claim 1, wherein the insertion depth is a distance from a lip of the patient to an end of the endotracheal tube when the endotracheal tube is positioned within a trachea of the patient.

9. A system, comprising:
a memory that stores computer executable components; and
a processor, operably coupled to the memory, and that executes the computer executable components stored in the memory, wherein the computer executable components comprise:
an insertion depth component that determines an insertion depth of an endotracheal tube by digitally measuring a nose to tragus length of a patient via an analysis of image data that characterizes an anatomy of the patient.

10. The system of claim 9, further comprising:
an image capturing device that captures the image data; and
a target component that identifies a first target reference point from the image data that correlates to a location of an ear of the patient and a second target reference point from the image data that correlates to a location of a nose of the patient.

11. The system of claim 10, further comprising:
a display that superimposes icons onto the image data at the first target reference point and the second target reference point.

12. The system of claim 10, further comprising:
a measurement component that measures a distance between the first target reference point and the second target reference point to generate a digitally measured nose to tragus length.

13. The system of claim 12, further comprising:
a correlation component that determines the insertion depth based on the digitally measured nose to tragus length in accordance with a defined relationship between the insertion depth and the nose to tragus length of the patient.

14. The system of claim 9, wherein the insertion depth is a distance from a lip of the patient to an end of the endotracheal tube when the endotracheal tube is positioned within a trachea of the patient.

15. A computer-implemented method, comprising:
determining, by a system operatively coupled to a processor, an insertion depth of an endotracheal tube by digitally measuring an NTL of a patient via an analysis of image data that characterizes an anatomy of the patient.

16. The computer-implemented method of claim 15, further comprising:
capturing, by the system, the image data; and
identifying, by the system, a first target reference point from the image data that correlates to a location of an ear of the patient and a second target reference point from the image data that correlates to a location of a nose of the patient.

17. The computer-implemented method of claim 16, further comprising:
superimposing, by the system, icons onto the image data at the first target reference point and the second target reference point.

18. The computer-implemented method of claim 16, further comprising:
measuring, by the system, a distance between the first target reference point and the second target reference point to generate a digitally measured nose to tragus length.

19. The computer-implemented method of claim 18, further comprising:
determining, by the system, the insertion depth based on the digitally measured nose to tragus length in accordance with a defined relationship between the insertion depth and the nose to tragus length of the patient.

20. The computer-implemented method of claim 15, wherein the insertion depth is a distance from a lip of the patient to an end of the endotracheal tube when the endotracheal tube is positioned within a trachea of the patient.
